(19) United States
(12) Patent Application Publication
(10) Pub. No.: US 2025/0331784 A1
(43) Pub. Date: Oct. 30, 2025

(54) Device for Wrist Radiography

(71) Applicant: THE UNIVERSITY OF WESTERN ONTARIO, London (CA)

(72) Inventors: Emily LALONE, London (CA); Maxwell CAMPBELL, London (CA)

(73) Assignee: THE UNIVERSITY OF WESTERN ONTARIO, London (CA)

(21) Appl. No.: 19/189,513

(22) Filed: Apr. 25, 2025

Related U.S. Application Data
(60) Provisional application No. 63/638,776, filed on Apr. 25, 2024.

(51) Int. Cl. A61B 6/04; A61G 13/12

(52) U.S. Cl. CPC A61B 6/0407; A61G 13/124; A61G 2210/50

(57) ABSTRACT

A wrist radiography device comprising: a body including (i) a straight bottom surface (horizontal), (ii) a first oblique surface angled at 45° relative to the horizontal, (iii) a second oblique surface angled 60° relative to the horizontal, and (iv) a vertical (90° relative to the horizontal) lateral surface. Also a method of positioning a wrist of a patient prior and/or during radiograph examination of the wrist using the wrist radiography device, and a kit comprising the wrist radiography device and instructions for positioning a wrist of a patient prior and during radiograph examination.

### CROSS REFERENCE TO RELATED APPLICATIONS

This application claims the benefit under 35 U.S.C. § 119(e) of U.S. Provisional Ser. No. 63/638,776, filed Apr. 25, 2024, the content of which is hereby incorporated by reference in its entirety into the present disclosure.

### FIELD OF TECHNOLOGY

The present disclosure relates to a device for wrist radiography.

### BACKGROUND INFORMATION

X-ray radiographic examination of the hand is performed using posteroanterior (PA), oblique and lateral projections. These projections are obtained by positioning the limb on the flat surface of the image detector at angles to optimize viewing of the overlapping bones in the hand and wrist in the subsequent X-ray images. Positioning errors are common to wrist radiographs. PA positioning is currently addressed through the use of adhesive tapes, multiple radiographic positioning sponges or through radiographer best judgement. Radiographic positioning sponges are used to position the wrist for oblique projections and come in standard degrees of inclination varying from 21°-60°. They are comprised of a low density closed cell foam with minimal x-ray attenuation and thus negligible artifact. Positioning sponges see limited clinical use due to high cost and limited shelf-space in the medical radiation suite. In the case that sponges are not used, anatomical positioning is done solely through radiographer best judgement and experience. There are standard positioning procedures used by radiographers to position the wrist, but there are high levels of variability in position between radiographers and variability in procedures between x-ray centres. This lack of standardization across the medical radiation field leads to high levels of variability in the diagnostic value of wrist x-rays. There are currently no clinically available positioning tools intended solely to aid in the posteroanterior projection of the wrist. Previous work has been done to develop a tool for lateral projection, positioning the dorsal surface of the hand perpendicular to the image detector [1]. These previous inventions use large, non-portable fixtures fabricated from glass and other radiopaque materials, leaving an artifact in the developed radiograph.

Current clinical instruction for PA wrist projection involves positioning the volar (palmar) surface of the wrist as the anatomical reference for the PA projection. Positioning the wrist using the volar surface is inherently variable due to differences in patient thenar eminence anatomy. The thenar eminence causes radial elevation (supination) of the wrist, rotating the carpal plane in reference to the incident x-ray beam.

Reducing positioning errors will increase the diagnostic value of wrist x-rays, making wrist radiography more repeatable, while reducing the need for repeat x-rays/or alternate medical imaging. Reducing the repeat x-ray rate will reduce the subsequent radiation dose to patients, medical waste, and the workload on medical radiation units. As such, there is a need for a device that reduces the positioning errors of the wrist during radiography.

### SUMMARY OF DISCLOSURE

Presented herein, in one embodiment, is a wrist radiography device comprising: a body including (i) a straight bottom surface (horizontal), (ii) a first oblique surface angled at 45° relative to the horizontal, (iii) a second oblique surface angled 60° relative to the horizontal, and (iv) a vertical (90° relative to the horizontal) lateral surface.

In one embodiment of the wrist radiography device of the present disclosure, the wrist radiography device further comprises a removable base having a cavity.

In another embodiment of the wrist radiography device of the present disclosure, the body is a truncated pyramid-shaped body.

In another embodiment of the wrist radiography device of the present disclosure, the truncated pyramid shaped body further includes an arm connected to a vertical axis for (i) concentrical movement about the vertical axis between a retracted position and an extended position out from body, and (ii) up and down reciprocal movement along the vertical axis.

In another embodiment of the wrist radiography device of the present disclosure, the arm includes a see-through opening.

In another embodiment of the wrist radiography device of the present disclosure, the arm of the truncated pyramid shaped body is removable.

In another embodiment of the wrist radiography device of the present disclosure, the arm of the truncated pyramid-shaped body includes a third oblique surface angled 30° relative to the horizontal.

In another embodiment of the wrist radiography device of the present disclosure, the body is a pyramid-shaped body.

In another embodiment of the wrist radiography device of the present disclosure, the pyramid-shaped body further includes an arm connected to a vertical axis for (i) concentrical movement about the vertical axis between a retracted position and an extended position out from pyramid-shaped body, and (ii) up and down reciprocal movement along the vertical axis.

In another embodiment of the wrist radiography device of the present disclosure, the arm includes a see-through opening.

In another embodiment of the wrist radiography device of the present disclosure, the arm is removable from the body.

In another embodiment of the wrist radiography device of the present disclosure, the device includes, or is made of, a polymer that has low contrast under x-ray beams.

In another embodiment, the present disclosure relates to a method of positioning a wrist of a patient prior and/or during radiograph examination of the wrist comprising: (i) providing a device according to an embodiment of the present disclosure, and (ii) performing at least one the following steps: a) placing a volar (palmar) or dorsal surface of the wrist parallel to the first oblique surface for a posteroanterior (PA) or anteroposterior (AP) oblique projection with a 45° rotation respectively, b) placing the volar (palmar) or dorsal surface of the wrist parallel to the second oblique surface for a PA or AP oblique projection with a 60° rotation respectively, c) placing the volar (palmar) or dorsal surface of the wrist parallel to the second oblique surface for a PA or AP oblique projection with a 30° rotation respectively, d) with the arm to the extended position, placing a dorsal surface of the wrist parallel to the arm for a posteroanterior projection, and/or e) placing the dorsal surface of the wrist parallel to the bottom surface or to the vertical lateral surface for a lateral projection.

In one embodiment of the method of positioning a wrist of a patient prior and/or during radiograph examination of the wrist of the present disclosure, the device is the device having the truncated pyramid-shaped body having the third oblique angled 30° relative to the horizontal, and wherein step c) comprises placing the volar (palmar) or dorsal surface of the wrist parallel to the third oblique surface of the arm of the truncated pyramid-shaped body for a PA or AP oblique projection with a 30° rotation respectively.

In another embodiment of the method of positioning a wrist of a patient prior and/or during radiograph examination of the wrist of the present disclosure, the device is the device having an arm having a see-through opening and the method further includes obtaining an x-ray image of the wrist through the see-through opening of the arm of the device.

In another embodiment of the method of positioning a wrist of a patient prior and/or during radiograph examination of the wrist of the present disclosure, step d) is performed with the patient's hand clenched for a PA clenched positioning.

In another embodiment, the present disclosure provides for a kit comprising a wrist radiography device according to an embodiment of the present disclosure and instructions for positioning a wrist of a patient prior and during radiograph examination.

### BRIEF DESCRIPTION OF THE DRAWINGS

The following figures illustrate various aspects and preferred and alternative embodiments.

FIG. 1A—Perspective view of a device for wrist radiology with a truncated pyramid-shaped body in accordance with one embodiment of the present disclosure.

FIG. 1B—Perspective view of a device for wrist radiology with a pyramid-shaped body in accordance with one embodiment of the present disclosure.

FIGS. 2A-2B—2A: Cross section of a device for wrist radiology with a truncated pyramid-shaped body in accordance with one embodiment of the present disclosure. 2B: Cross section of a device for wrist radiology with a pyramid-shaped body in accordance with one embodiment of the present disclosure.

FIGS. 3A-3B—3A: Cross section of a device for wrist radiology with a truncated pyramid-shaped body in accordance with one embodiment of the present disclosure. 3B: Cross section of a device for wrist radiology with a pyramid-shaped body in accordance with one embodiment of the present disclosure.

FIGS. 4A-4B—4A: Top view of a device for wrist radiology with a truncated pyramid-shaped body in accordance with one embodiment of the present disclosure having its arm extended. 4B: Top view of a device for wrist radiology with a pyramid-shaped body in accordance with one embodiment of the present disclosure having its arm extended.

FIGS. 5A-5C—5A: Overhead perspective view of a device with a truncated pyramid-shaped body of the present disclosure in use for anteroposterior (AP) oblique projections (30°/60°/45°) in accordance with one embodiment. 5B: Overhead perspective view of a device with a pyramid-shaped body of the present disclosure in use for anteroposterior (AP) oblique projections (30°/60°/45°) in accordance with one embodiment. 5C: Overhead view of the arm removed from a device with a truncated pyramid-shaped body of the present disclosure in use for anteroposterior (AP) oblique projections (30°) in accordance with one embodiment.

FIGS. 6A-6C—6A: Front view of a device with a truncated pyramid-shaped body of the present disclosure in use for AP oblique projections (30°/60°/45°) in accordance with one embodiment. 6B: Front view of a device with a pyramid-shaped body of the present disclosure in use for AP oblique projections (with a 30°/60°/45°rotation) in accordance with one embodiment. 6C: Front view of the arm removed from a device with a truncated pyramid-shaped body of the present disclosure in use for AP oblique projections (with a 30° rotation) in accordance with one embodiment.

FIGS. 7A-7C—7A: Overhead view of a device with a truncated pyramid-shaped body of the present disclosure in use for posteroanterior (PA) oblique projections (30°/60°/45°) in accordance with one embodiment. 7B: Overhead view of a device with a pyramid-shaped body of the present disclosure in use for posteroanterior (PA) oblique projections (30°/60°/45°) in accordance with one embodiment. 7C: Overhead view of the arm of a device with a truncated pyramid-shaped body of the present disclosure in use for posteroanterior (PA) oblique projections (30°) in accordance with one embodiment.

FIGS. 8A-8C—8A: Front view of a device of the present disclosure with a truncated pyramid-shaped body in use for PA oblique projections (30°/60°/45°) in accordance with one embodiment. 8B: Front view of a device of the present disclosure with a pyramid-shaped body in use for PA oblique projections (30°/60°/45°) in accordance with one embodiment. 8C: Front view of the arm of a device with a truncated pyramid-shaped body of the present disclosure in use for PA oblique projections (30°) in accordance with one embodiment.

FIGS. 9A-9B—9A: Overhead view of a device of the present disclosure with a truncated pyramid-shaped body in use for PA positioning in accordance with one embodiment. 9B: Overhead view of a device of the present disclosure with a pyramid-shaped body in use for PA positioning in accordance with one embodiment.

FIGS. 10A-10B—10A: Front view of a device of the present disclosure with a truncated pyramid-shaped body in use for PA positioning in accordance with one embodiment. 10B: Front view of a device of the present disclosure with a pyramid-shaped body in use for PA positioning in accordance with one embodiment.

FIGS. 11A-11B—10A: Overhead view of a device of the present disclosure with a truncated pyramid-shaped body in use for PA (clenched) positioning in accordance with one embodiment. 10B: Overhead view of a device of the present disclosure with a pyramid-shaped body in use for PA (clenched) positioning in accordance with one embodiment.

FIGS. 12B to 12B—12A: Front view of a device of the present disclosure with a truncated pyramid-shaped body in use for PA (clenched) positioning in accordance with one embodiment. 12B: Front view of a device of the present disclosure with a pyramid-shaped body in use for PA (clenched) positioning in accordance with one embodiment.

FIGS. 13A-13B—13A: Overhead view of a device of the present disclosure with a truncated pyramid-shaped body in use for lateral positioning in accordance with one embodiment. 13B: Overhead view of a device of the present disclosure with a pyramid-shaped body in use for lateral positioning in accordance with one embodiment.

FIGS. 14A-14B—14A: Front view of a device of the present disclosure with a truncated-pyramid-shaped body in use for lateral positioning in accordance with one embodiment. 14B: Front view of a device of the present disclosure with a truncated-pyramid-shaped body in use for lateral positioning in accordance with one embodiment.

### DETAILED DISCLOSURE

### Definitions

In this specification and in the claims that follow, reference will be made to several terms that shall be defined to have the meanings below. All numerical designations, e.g., dimensions and weight, including ranges, are approximations that typically may be varied (+) or (−) by increments of 0.1, 1.0, or 10.0, as appropriate. All numerical designations may be understood as preceded by the term “about”.

The term “about,” particularly in reference to a given quantity, is meant to encompass deviations of plus or minus five percent.

As used in the specification and claims, the singular form “a”, “an” and “the” include plural references unless the context clearly dictates otherwise. For example, the term “a compound” includes a plurality of compounds, including mixtures thereof.

As used herein, the terms “comprising,” “including,” “having” are intended to mean that the devices and methods include the recited elements, but do not exclude others. “Consisting essentially of” when used to define devices and methods, shall mean allowing only those additional elements that do not materially affect the invention's basic and novel characteristics. “Consisting of” shall mean excluding any element not specified in a claim. Embodiments defined by each of these transition terms are within the scope of this disclosure.

A “truncated pyramid” means a pyramid cut at a plane parallel to the base and separating the part containing the pyramid's apex.

### Table of Reference Numbers

10: wrist radiography device.
11: body. Reference number “11” is used in the description, but not in the Figures, to refer to both the truncated pyramid shaped body 11a and to the complete pyramid-shaped body 11b.
11a: truncated pyramid-shaped body.
11b: complete pyramid-shaped body (in this document, unless specified otherwise, the term “pyramid-shaped body” refers to the “complete pyramid-shaped body”).
12: bottom surface.
13: first oblique surface.
14: second oblique surface.
15: oblique surface on arm 17a.
16: vertical lateral surface.
17a: arm of truncated-pyramid shaped body 11a.17b: arm of complete pyramid-shaped body 11b.
18: vertical axis (column or shaft).
19: opening included in the arm 17a,b.
20: base (storage).
21: cavity in base 20.
22: tool for patient to grasp.
23: vertical lateral surface.
24: cutout that allows reciprocal movement of arm 17a,b.
25: cavity in truncated pyramid-shaped body 11a to house arm.

### Overview

The present disclosure relates to a wrist radiography device that reduces the positioning errors of the wrist during x-ray radiographs. The device of the present disclosure is to be used within the clinical environment to aid radiation technologists' repeatably position the wrist for all standard radiographic views. A single wrist radiography device of the present disclosure is used for all standard radiographic wrist views, the lateral, oblique, and posteroanterior (PA) projection, thereby avoiding the use of multiple sponge devices. The device of the present disclosure includes an arm that provides a novel positioning technique for the PA wrist projection.

The arm positions the dorsal surface of the wrist, eliminating complications from variable patient thenar eminences, consistently placing the carpal plane perpendicular to the incident x-ray beam.

### The Wrist Radiography Device

With reference to the figures, in one embodiment, a wrist radiography device 10 comprises a body (truncated pyramid-shaped 11a or complete pyramid-shaped 11b), with five flat surfaces including (i) a straight bottom surface 12, (ii) a first oblique surface 13 angled at 45° relative to the bottom surface 12, (iii) a second oblique surface 14 angled 60° relative to the bottom surface 12, and (iv) two vertical surfaces 16,23 angled at 90° relative to the bottom surface 12. The bottom surface 12 is used for convenience in order to describe the relative position and angles of the surfaces with respect to each other. All surfaces are flat, and any surface can be laid onto the flat surface of the X-ray image detector in order to position a limb against another surface of the device to achieve a desired position. The bottom surface 12 is a rectangle with two short sides and two long sides. The two long sides comprise one flat (90°) surface 23, and one oblique (60/30°) surface 14. The two short sides comprise one flat (90°) surface 16, and one oblique (45°) surface 13.

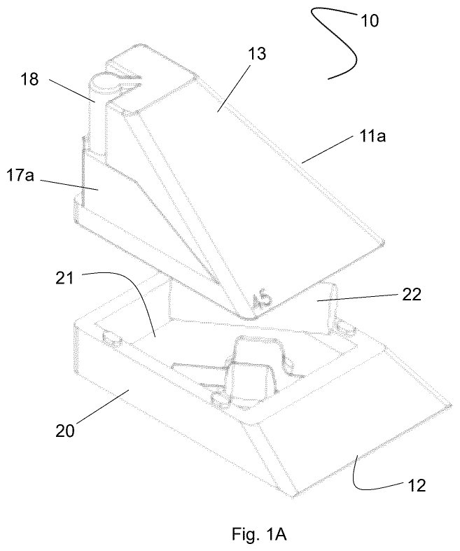

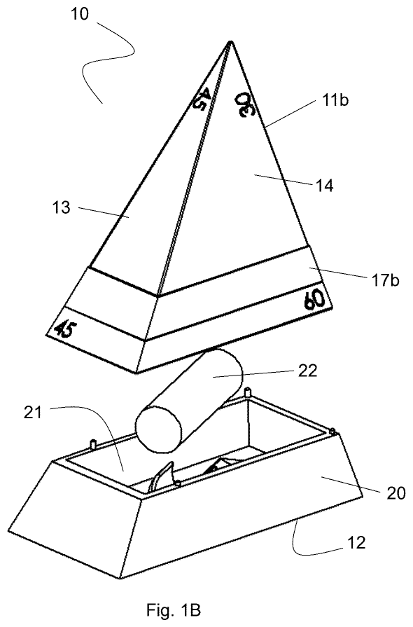

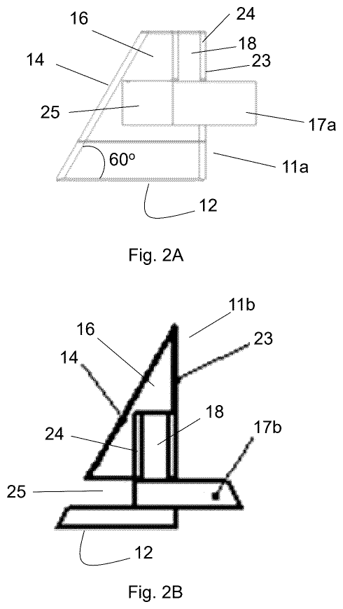

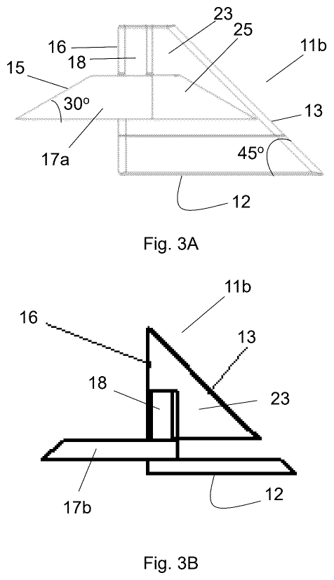

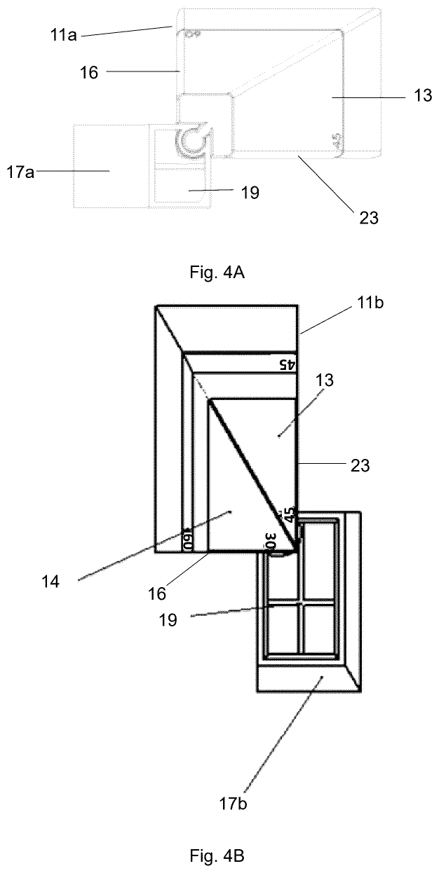

In one embodiment, the body 11a,b of device 10 further includes an arm 17a,b that extends out from the body 11a,b. In one embodiment the arm 17a,b is connected to a vertical axis 18, which may take the form of a shaft or column, for (i) concentric movements about the vertical axis 18 between a retracted position and an extended position out from the body 11a,b, and (ii) up and down reciprocal movement along the vertical axis 18. In one embodiment, vertical shaft 18 is recessed within the body 11a,b. FIGS. 1A and 1B show arm 17a (FIG. 1A) and 17b (FIG. 1B) in a retracted position and FIGS. 2A-2B and 3A-3B show arm 17A (FIGS. 2A and 3A) and arm 17b (FIGS. 2B and 3B) in an extended position. The body 11a,b includes a cutout 24 out of the edge between the lateral surfaces 16, 23 that houses the vertical axis 18. Cutout 24 allows the arm 17a,b to move up and down along the vertical axis 18. In one embodiment, arm 17a,b includes a see-through means 19 that allows to see through the arm 17a,b. In another embodiment, arm 17a,b includes an opening 19. Opening 19 allows radiographers to visualize the anatomy they are palpating and for correct alignment of the incident x-ray beam with the major and minor axes of the wrist. In another embodiment, arm 17a,b is made of an x-ray transparent material for allowing radiographers to visualize the anatomy they are palpating and for correct alignment of the incident x-ray beam with the major and minor axes of the wrist.

In one embodiment, the body of device 10 takes the form of a truncated pyramid-shaped body 11a. The arm 17a is removable from the body 11a. The arm 17a of truncated pyramid-shaped body 11a includes (v) an oblique surface 15 angled 30° relative to the horizontal. In this document, the oblique surface 15 is referred to alternatively as the third oblique surface or as the oblique surface of arm 17a.

In another embodiment, the body of device 10 takes the form of a pyramid-shaped body 11b. In one embodiment, the arm 17b is removable from the body 11b.

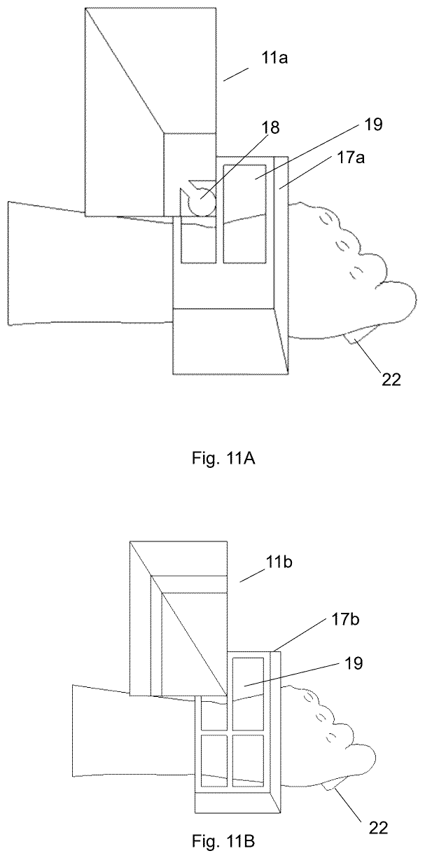

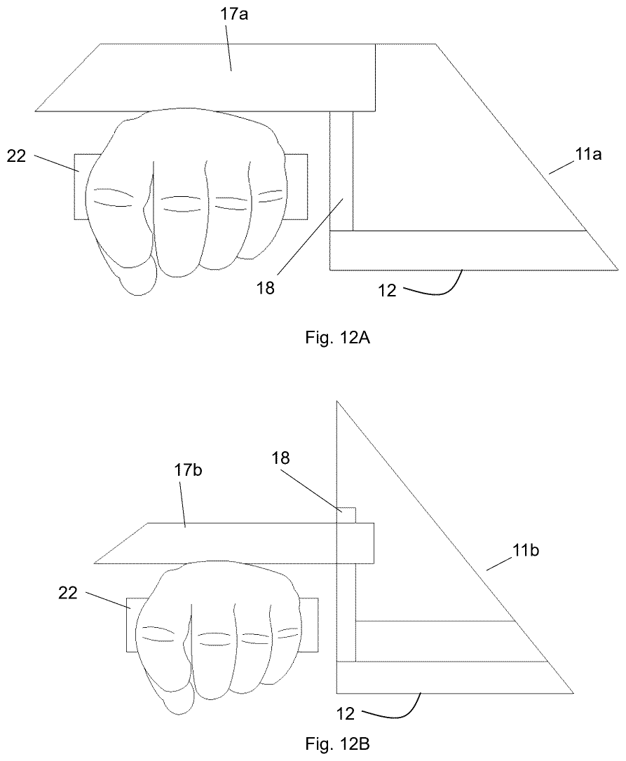

In one embodiment, the body 11a,b further includes a removable base 20. Base 20, in one embodiment, includes a cavity 21 which houses one or more tools, such as a tool 22 for a patient to grasp to obtain a PA clench position (FIGS. 11A-11B and 12A-12B).

In one embodiment, the body 11a,b includes a cavity 25 that houses the arm 17a,b when not in use.

The device 10 of the present disclosure can be made by any suitable manufacturing method. For example, in one embodiment, device 10 is 3D printable to aid in technology dissemination and conforms to “print in place” principles to simplify the assembly process. With 3D printing no assembly of the tool 10 is required. In another embodiment, device 10 is injection mouldable, rotomould, machinable, and/or castable.

In another embodiment, device 10 includes an internal structure having low contrast under x-ray beams. In one embodiment, device 10 is configured such that the interaction between the incident x-ray beam and the device's 10 infill are minimized, thereby reducing the artifact on developed radiographs. Device 10 includes a geometry that minimizes the interaction between the incident x-ray beam and the device's 10 infill.

In another embodiment, device 10 is made of a polymer that has low contrast under x-ray beams, such as polylactic acid (PLA).

### Methods of Using the Wrist Radiography Device

In another embodiment, the present disclosure relates to a method of positioning a wrist of a patient prior and/or during radiograph examination of the wrist using a wrist radiography device of the present disclosure. Placing a surface of the wrist parallel to a surface of the device can be achieved, for example, by placing the wrist in contact with that surface of the device.

Oblique positioning is achieved by placing the volar (palmar) or dorsal surface of the wrist parallel to one of the angled oblique surfaces 13, 14 or 15..

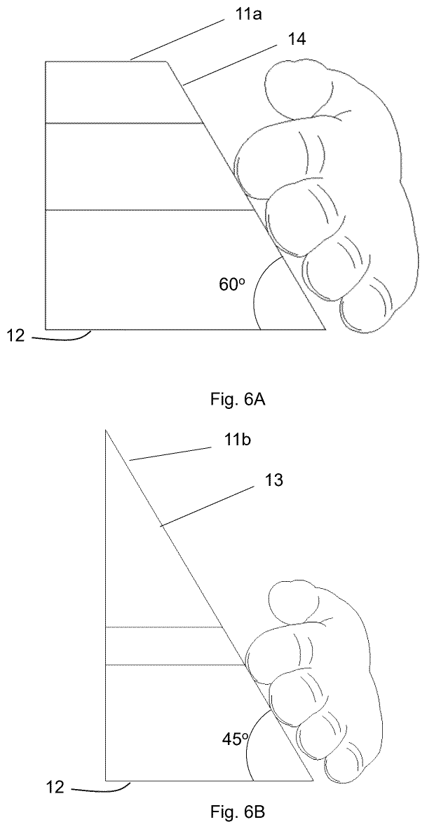

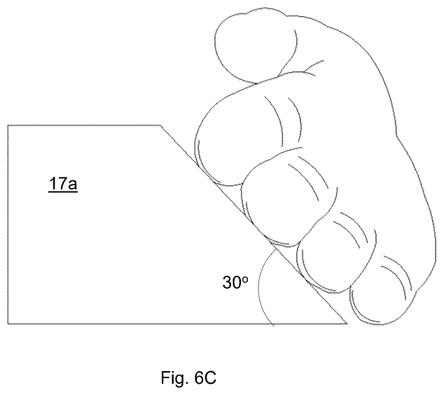

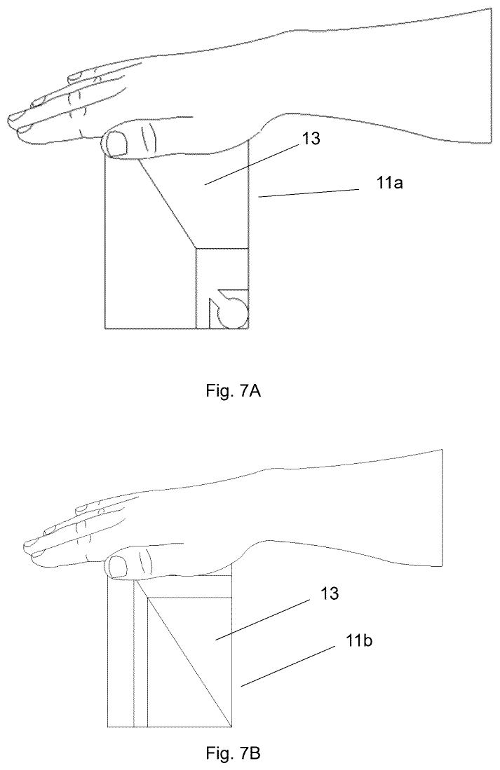

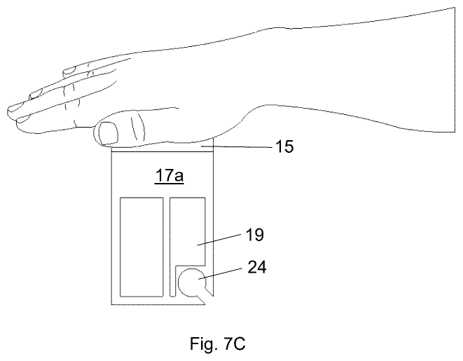

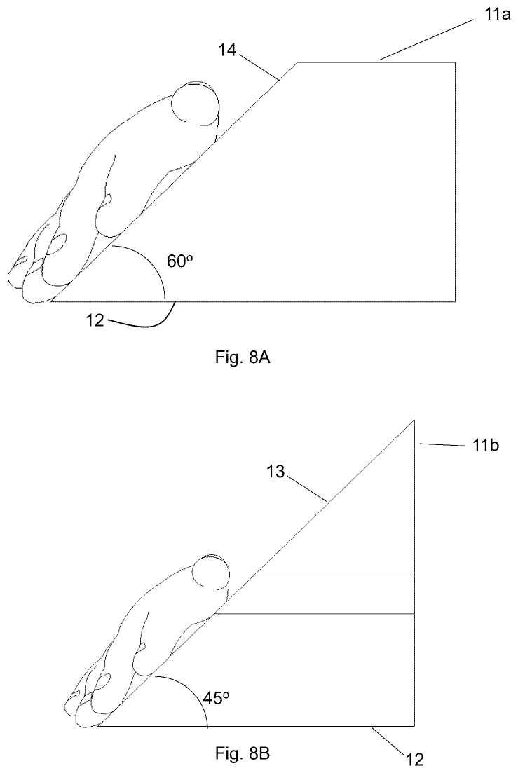

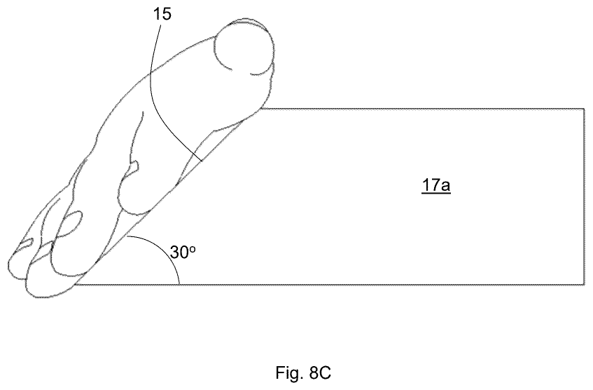

The first surface 13 of the body 11a,b angled at 45° to the horizontal (i.e., 45° relative to the straight bottom surface 12 of the body 11a,b) and the image detector, is used for AP oblique projections with a 45° rotation (FIG. 6B) and PA oblique projections with a 45° rotation (FIGS. 7A and 8B) oblique projections.

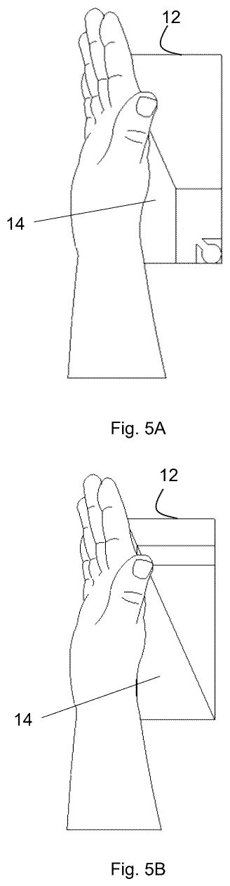

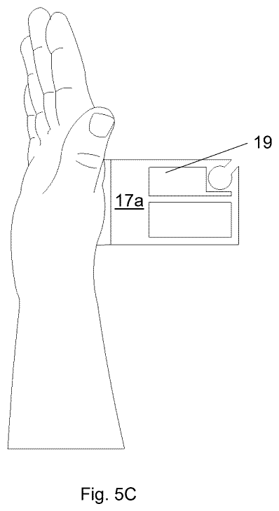

The second surface 14 of the body 11a,b angled at 60° to the horizontal and the image detector is used for AP oblique projections with 30° and 60° rotations (FIGS. 5A, 5B, and 6A) and AP oblique projections with 30° and 60° rotations (FIG. 8A).

Arm 17a is removable from body 11a. As such, in one embodiment, arm 17a is removed from body 11a and the third surface 15 of the arm 17a angled at 30° to the horizontal and the image detector is used for AP oblique projections with a 30° rotation (FIGS. 5C and 6C) and PA oblique projections with a 30° rotation (FIGS. 7C and 8C).

In another embodiment, the second surface of the pyramid-shaped body 11b angled at 60° to the horizontal can also be used for PA and AP oblique projections with a 30° rotation by flipping the body 11b.

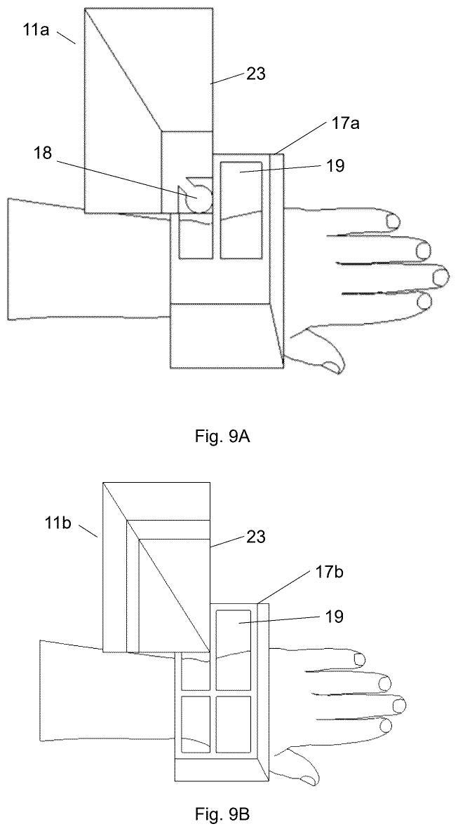

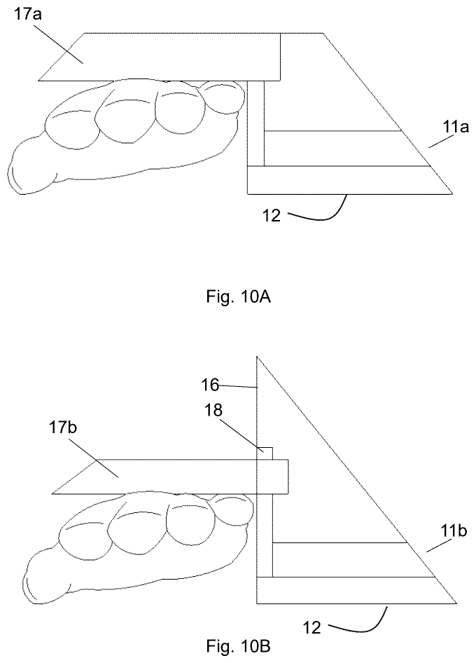

A posteroanterior (PA) projection is achieved using the arm 17 of the body 11. The PA position is achieved by placing the dorsal surface of the wrist parallel to the arm 17 (FIGS. 9 to 12). This PA position places the carpal plane of the wrist parallel to the image detector and accommodates for differences in each patient's thenar eminence. The arm 17 extends out of the base 12 and translates concentrically about a recessed column 18 within the base 12 of the device 10. The arm 17a can be removed from the truncated pyramid-shaped body 11a if translated superior to the base 12 along the recessed column 18. The arm 17 contains the opening or window 19, which allows radiographers to visualize the anatomy they are palpating and for correct alignment of the incident beam with the major and minor axes of the wrist. The arm 17a contains an oblique surface 15 angled at 30° to the horizontal and image detector that is used for a PA or anteroposterior (AP) oblique projection with a 30° rotation.

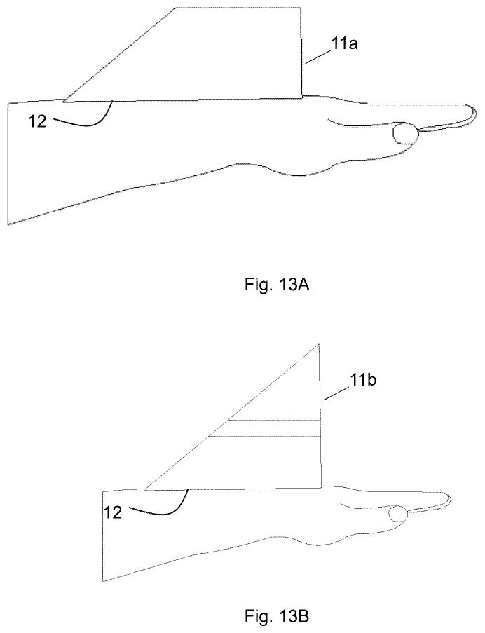

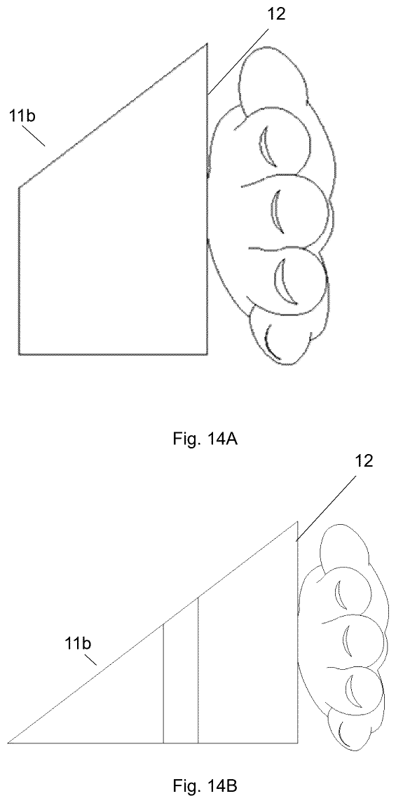

With reference to FIGS. 13 and 14, the lateral projection is achieved by placing the dorsal surface of the wrist perpendicular (90°) to the image detector by placing parallel 10 to either the bottom surface 12 or to one of the lateral surfaces 16 or 23, when positioned perpendicular to the image detector.

As such, in another embodiment, the present disclosure provides for a method of positioning a wrist of the patient prior to and/or during radiograph examination of the wrist comprising providing the device 10 and performing at least one the following steps:

a) placing a volar (palmar) or dorsal surface of the wrist parallel to the first oblique surface for a PA or AP oblique projection with a 45° rotation, respectively,
b) placing the volar (palmar) or dorsal surface of the wrist parallel to the second oblique surface for a PA or AP oblique projection with a 30° or a 60° rotation, respectively,
c) placing the volar (palmar) or dorsal surface of the wrist parallel to the third oblique surface for a PA or AP oblique projection with a 30° rotation, respectively,
d) with the arm to the extended position, placing a dorsal surface of the wrist parallel to the arm for a posteroanterior projection, and/or
e) placing the dorsal surface of the wrist parallel to the bottom surface or to the vertical lateral surface for a lateral projection.

In one embodiment, step d) is performed with the patient's hand clenched for a PA clenched positioning. The patient can hold something in the patient's hands (such as tool 22) to squeeze down on.

In another embodiment, the present disclosure provides for a kit comprising a wrist radiography device according to an embodiment of the present disclosure and instructions on how to use the device to position a wrist of a patient prior and during radiograph examination.

### REFERENCES

[1] Larsen, C. Falck, et al. “Radiography of the wrist: A new device for standardized radiographs.” Acta Radiologica 31.5 (1990): 459-462.

Although various embodiments of the disclosure have been described and illustrated, it will be apparent to those skilled in the art in light of the present description that numerous modifications and variations can be made. The scope of the invention is defined more particularly in the appended claims.

What is claimed is:

1. A wrist radiography device comprising: a body including (i) a bottom surface (horizontal), (ii) a first oblique surface angled at 45° relative to the horizontal, (iii) a second oblique surface angled 60° relative to the horizontal, and (iv) a vertical (90° relative to the horizontal) lateral surface.

2. The wrist radiograph device of claim 1, wherein the wrist radiography device further comprises a removable base having a cavity.

3. The wrist radiography device of claim 1, wherein the body is a truncated pyramid-shaped body.

4. The wrist radiography device of claim 3, wherein the truncated pyramid shaped body further includes an arm connected to a vertical axis for (i) concentrical movement about the vertical axis between a retracted position and an extended position out from body, and (ii) up and down reciprocal movement along the vertical axis.

5. The wrist radiography device of claim 4, wherein the arm includes a see-through opening.

6. The wrist radiography device of claim 4, wherein the arm of the truncated pyramid shaped body is removable.

7. The wrist radiography device of claim 4, wherein the arm of the truncated pyramid-shaped body includes a third oblique surface angled 30° relative to the horizontal.

8. The wrist radiography device of claim 1, wherein the body is a pyramid-shaped body.

9. The wrist radiography device of claim 8, wherein the pyramid-shaped body further includes an arm connected to a vertical axis for (i) concentrical movement about the vertical axis between a retracted position and an extended position out from pyramid-shaped body, and (ii) up and down reciprocal movement along the vertical axis.

10. The wrist radiography device of claim 9, wherein the arm includes a see-through opening.

11. The wrist radiography device of claim 9, wherein the arm is removable from the body.

12. The wrist radiography device of claim 1, wherein the device includes a polymer that has low contrast under x-ray beams.

13. A method of positioning a wrist of a patient prior and/or during radiograph examination of the wrist comprising: (i) providing the device of claim 1, and (ii) performing at least one the following steps:
a) placing a volar (palmar) or dorsal surface of the wrist parallel to the first oblique surface for a posteroanterior (PA) or anteroposterior (AP) oblique projection with a 45° rotation respectively,
b) placing the volar (palmar) or dorsal surface of the wrist parallel to the second oblique surface for a PA or AP oblique projection with a 60° rotation respectively,
c) placing the volar (palmar) or dorsal surface of the wrist parallel to the second oblique surface for a PA or AP oblique projection with a 30° rotation respectively,
d) with the arm to the extended position, placing a dorsal surface of the wrist parallel to the arm for a posteroanterior projection, and/or
e) placing the dorsal surface of the wrist parallel to the bottom surface or to the vertical lateral surface for a lateral projection.

14. The method of claim 13, wherein the body of the device is a truncated-pyramid shaped body having an arm connected to a vertical axis for (i) concentrical movement about the vertical axis between a retracted position and an extended position out from body, and (ii) up and down reciprocal movement along the vertical axis and the arm includes a third oblique surface angled 30° relative to the horizontal, and wherein step c) comprises placing the volar (palmar) or dorsal surface of the wrist parallel to the third oblique surface of the arm of the truncated pyramid-shaped body for a PA or AP oblique projection with a 30° rotation respectively.

15. The method of claim 13, wherein the body of the device is a truncated-pyramid shaped body having an arm connected to a vertical axis for (i) concentrical movement about the vertical axis between a retracted position and an extended position out from body, and (ii) up and down reciprocal movement along the vertical axis and the arm includes a see-through opening, and wherein the method further includes obtaining an x-ray image of the wrist through the see-through opening of the arm.

16. The method of claim 13, wherein the body of the device is a pyramid-shaped body having an arm connected to a vertical axis for (i) concentrical movement about the vertical axis between a retracted position and an extended position out from pyramid-shaped body, and (ii) up and down reciprocal movement along the vertical axis and the arm includes a see-through opening, and wherein the method further includes obtaining an x-ray image of the wrist through the see-through opening of the arm.

17. The method of claim 13, wherein step d) is performed with the patient's hand clenched for a PA clenched positioning.

18. A kit comprising the wrist radiography device of claim 1 and instructions for positioning a wrist of a patient prior and during radiograph examination.
